(19) United States
(12) Patent Application Publication
(10) Pub. No.: US 2019/0047929 A1
(43) Pub. Date: Feb. 14, 2019

(54) PROCESS FOR THE HYDROGENATION OF GLYCOLALDEHYDE

(71) Applicant: SHELL OIL COMPANY, HOUSTON, TX (US)

(72) Inventors: Dionysius Jacobus Maria DE VLIEGER, Amsterdam (NL); Pieter HUIZENGA, Amsterdam (NL); Evert VAN DER HEIDE, Amsterdam (NL); Smita EDULJI, Houston, TX (US); Jean Paul Andre Marie Joseph Ghislain LANGE, Amsterdam (NL)

(21) Appl. No.: 16/075,935

(22) PCT Filed: Feb. 6, 2017
(86) PCT No.: PCT/EP2017/052546

§ 371 Date: Aug. 6, 2018

(30) Foreign Application Priority Data
Feb. 8, 2016 (EP) 16154670.0

(51) Int. Cl. C07C 29/141; C07C 45/60

(52) U.S. Cl. CPC C07C 29/141; C07C 45/60

(57) ABSTRACT

The invention provides a process for the selective hydrogenation of glycolaldehyde in a process stream comprising glycolaldehyde and one or more monosaccharide in a solvent, said process comprising contacting the process stream with hydrogen in the presence of a hydrogenation catalyst composition at a temperature of no more than 150° C. and for a residence time of no more than 90 minutes.

### FIELD OF THE INVENTION

The present invention relates to a process for the selective hydrogenation of glycolaldehyde.

### BACKGROUND OF THE INVENTION

Monoethylene glycol (MEG) and monopropylene glycol (MPG) are valuable materials with a multitude of commercial applications, e.g. as heat transfer media, antifreeze, and precursors to polymers, such as polyethylene terephthalate (PET). MEG and MPG are typically made on an industrial scale by hydrolysis of the corresponding alkylene oxides, which are the oxidation products of ethylene and propylene, produced from fossil fuels.

In recent years, increased efforts have focussed on producing chemicals, including glycols, from renewable feedstocks, such as sugar-based materials. The conversion of sugars to glycols can be seen as an atom-efficient use of the starting materials with the oxygen atoms remaining intact in the desired product.

Current methods for the conversion of saccharides to glycols revolve around a retro-aldol/hydrogenation process as described in Angew. Chem. Int. Ed. 2008, 47, 8510-8513. Development of this technology has been on-going.

It is clearly desirable to maximise the yields of MEG and MPG in such processes and to deliver a process that can be carried out in a commercially viable manner. The market for MEG is generally more valuable than that for MPG, so a process particularly selective toward MEG would be advantageous.

A preferred methodology for a commercial scale process would be to use continuous flow technology, wherein feed is continuously provided to a reactor and product is continuously removed therefrom. By maintaining the flow of feed and the removal of product at the same levels, the reactor content remains at a more or less constant volume. Continuous flow processes for the production of glycols from saccharide feedstock have been described in US20110313212, CN102675045, CN102643165, WO2013015955 and CN103731258.

Processes for the conversion of saccharides to glycols generally require two catalytic species in order to catalyse the retro-aldol and hydrogenation reactions. The catalyst compositions used for the hydrogenation reactions tend to be heterogeneous. However, the catalyst compositions suitable for the retro-aldol reactions are generally homogeneous in the reaction mixture. Such homogeneous catalysts are inherently limited due to solubility constraints.

In general, ‘one-pot’ processes have been described. In these processes, the feed is contacted with both a retro-aldol and a hydrogenation catalyst at the same time. This adds complexity to the process in order to ensure that the correct balance of catalyst and feed ratios are maintained. Such processes may lead to high levels of impurities and undesired products.

It is known that thermal degradation of reaction intermediates, such as glycolaldehyde, can occur in the conversion of saccharides to glycols. Such degradation reduces the overall yield of desired products and increases the complexity of the isolation process of said desired products. It has generally been found that carrying out the reaction with high concentrations of starting materials in a reactor exacerbates this degradation and the formation of by-products.

Typically, the conversion of saccharides to glycols has, therefore, been carried out as a continuous flow process with a high degree of back mixing using a saccharide-containing feedstock comprising a low concentration of saccharide in solvent.

The balance between the retro-aldol and hydrogenation reactions has also been considered in detail. Typical by-products of saccharides to glycols processes are sugar alcohols. These include sorbitol, the hydrogenation product from glucose; xylitol, the hydrogenation product from xylose; and erythritol/threitol, hydrogenation products of C₄ monosaccharides. Sorbitol and other sugar alcohols are not suitable starting materials for the retro-aldol reactions to make glycolaldehyde, which can be reduced to MEG. Therefore, production of such sugar alcohols reduces the overall yield of MEG.

For this reason, and others, processes in which the retro-aldol and hydrogenation parts of the saccharides to glycols process are not carried out in an entirely concurrent manner have been described in the art.

In CN102731258, there is described a reactor in which there is suspended a catalyst filter basket in a position higher than the level of liquid reagents. The reagents are injected into the catalyst basket where they are contacted with hydrogenation catalyst compositions and then travel through the stirred slurry reactor in the bottom of the reactor vessel before flowing out of the bottom of the reactor. Said reactor vessel is equipped with a recycle loop from which reagents are re-injected into the catalyst basket.

US20150329449 describes a process in which a carbohydrate-containing feed is provided to a first reactor zone in which it is contacted with mainly retro-aldol catalyst. The feed is then provided to at least one further reaction zone containing a hydrogenation catalyst. In a preferred method described in US20150329449, the reactor chosen is a CSTR that contains a porous catalyst “basket” that is suspended in the reactor. The basket contains solid hydrogenation catalyst and occupies approximately 2% of the liquid volume of the reactor. In this operation the raw material is added to the reactor in such a way that the feed initially contacts the basket-free part of the reactor, before the stirring brings the reaction mixture into contact with the solid hydrogenation catalyst.

A particularly effective method of separating the retro-aldol and hydrogenation steps is taught in co-pending application EP15198769.0. This method requires a reactor system comprising a reactor vessel equipped with an external recycle loop. Saccharide-containing starting material and retro-aldol catalyst are provided to the recycle loop. As the starting material passes through the recycle loop with a short residence time, the retro-aldol reactions occur. The products of the retro-aldol reactions are then subjected to hydrogenation in the presence of a solid catalyst composition supported in the reactor vessel. A portion of the product stream is removed from the reactor vessel and the remainder is recycled back, via the recycle loop.

Recycle of a portion of the product stream allows dilution of the starting material stream and efficient recycle of at least a portion of the retro-aldol catalyst composition.

The presence of contaminants in saccharide-containing feedstocks is known to have a deactivating effect on the catalysts used in the conversion of such feedstocks to glycols. Severe deactivation may be caused by the presence of sulfur-containing contaminants, such as sulfur-containing amino acids (cysteine and methionine). A method to overcome this problem is described in co-pending application EP15174653.4 in which a starch feedstock is hydrolysed and the hydrolysed products are subjected to purification steps in order to remove sulfur-containing (and other) contaminants.

Further optimisation of a process for the conversion of saccharides into glycols is always desirable. It would be preferable to carry out a continuous process to provide glycols, and particularly MEG, from saccharide-containing feedstock in as high a yield as possible, while maintaining catalyst activity.

### SUMMARY OF THE INVENTION

Accordingly, the present invention provides a process for the selective hydrogenation of glycolaldehyde in a process stream comprising glycolaldehyde and one or more monosaccharide in a solvent, said process comprising contacting the process stream with hydrogen in the presence of a hydrogenation catalyst composition at a temperature of no more than 150° C. and for a residence time of no more than 90 minutes.

The present invention also provides a continuous process for the preparation of monoethylene glycol from starting material comprising one or more saccharides by:
i) contacting a feed stream comprising said starting material in a solvent with a retro-aldol catalyst composition in a first reaction zone at a temperature in the range of from 160 to 270° C. to provide an intermediate process stream comprising one or more monosaccharide and glycolaldehyde in a solvent;
ii) then contacting said intermediate process stream with hydrogen in the presence of a hydrogenation catalyst composition in a second reaction zone at a temperature of no more than 150° C. and for a residence time of no more than 90 minutes;
iii) withdrawing a product stream comprising glycols and one or more monosaccharide from the second reaction zone;
iv) providing a portion of said product stream for separation and purification of the glycols contained therein; and
v) recycling the rest of the product stream to the first reaction zone.

### BRIEF DESCRIPTION OF THE DRAWINGS

FIGS. 1 to 3 are schematic diagrams of exemplary, but non-limiting, embodiments of the process as described herein.

### DETAILED DESCRIPTION OF THE INVENTION

The present inventors have surprisingly found that the selective hydrogenation of glycolaldehyde may be carried out in the presence of one or more monosaccharide by carrying out the hydrogenation step at a temperature of no more than 150° C. and for a residence time of no more than 90 minutes. This process avoids the formation of sugar alcohols, which are unsuitable starting materials for a retro-aldol reaction.

Carrying out the hydrogenation step at such a low temperature also provides the added advantage that the hydrogenation catalysts used may tolerate sulfur-containing contaminants without any significant deactivation.

The selective hydrogenation of the present invention is particularly suitable in a continuous process for the preparation of monoethylene glycol from starting material comprising one or more saccharides. In said process the starting material may be subjected to a retro-aldol reaction and then the reactive intermediates thus formed subjected to a hydrogenation reaction. In such a process, it may be preferable or practical for the retro-aldol reaction not to proceed to completion before the reaction mixture is subjected to the hydrogenation step. The reaction mixture (or intermediate stream) at this stage will, therefore, comprise both glycolaldehyde and one or more monosaccharide. It is highly desirable to provide a process in which the glycolaldehyde in this intermediate stream is converted to monoethylene glycol without the one or more monosaccharide present being hydrogenated to sugar alcohols, non-useful by-products. The one or more monosaccharide may then be recycled to the retro-aldol reaction and the overall yield and selectivity of the reaction may be increased.

The present process is applied to a process stream comprising glycolaldehyde and one or more monosaccharide in a solvent. Any such process stream is suitable. A particularly preferred process stream is an intermediate stream in a process for the preparation of monoethylene glycol from starting material comprising one or more saccharides.

Said starting material preferably comprises at least one saccharide selected from the group consisting of monosaccharides, disaccharides, oligosaccharides and polysaccharides.

Saccharides, also referred to as sugars or carbohydrates, comprise monomeric, dimeric, oligomeric and polymeric aldoses, ketoses, or combinations of aldoses and ketoses, the monomeric form comprising at least one alcohol and a carbonyl function, being described by the general formula of CₙH₂ₙOₙ (n=4, 5 or 6). Typical C₄ monosaccharides comprise erythrose and threose, typical C₅ saccharide monomers include xylose and arabinose and typical C₆ sugars comprise aldoses like glucose, mannose and galactose, while a common C₆ ketose is fructose. Examples of dimeric saccharides, comprising similar or different monomeric saccharides, include sucrose, maltose and cellobiose. Saccharide oligomers are present in corn syrup. Polymeric saccharides include cellulose, starch, glycogen, hemicellulose, chitin, and mixtures thereof.

If said starting material comprises oligosaccharides or polysaccharides, it is preferable that it is subjected to pre-treatment before being fed to the reactor in a form that can be converted in the process of the present invention. Suitable pre-treatment methods are known in the art and one or more may be selected from the group including, but not limited to, sizing, drying, grinding, hot water treatment, steam treatment, hydrolysis, pyrolysis, thermal treatment, chemical treatment, biological treatment. However, after said pre-treatment, the starting material still comprises mainly monomeric and/or oligomeric saccharides. Said saccharides are, preferably, soluble in the reaction solvent.

Preferably, the starting material supplied to the reactor system after any pre-treatment comprises saccharides selected from starch and/or hydrolysed starch. Hydrolysed starch comprises glucose, sucrose, maltose and oligomeric forms of glucose. Said saccharide is suitably present as a solution, a suspension or a slurry in the solvent.

In one embodiment of the invention, the starting material also comprises sulfur-containing contaminants. Such sulfur-containing contaminants are typically present in the range of at most 1000 ppmw (based on the amount of sulfur, considered as the element, in the starting material (i.e. the carbohydrate or saccharide). Preferably the sulfur-containing contaminants are present in the range of at most 600 ppmw.

Optionally, little or no sulfur-containing contaminants are present, but in a typical process in which the feed comprises starch and/or hydrolysed starch and also comprises sulfur-containing contaminants, said sulfur-containing contaminants are typically present in the range of at least 10 ppmw (based on the amount of sulfur, considered as the element, in the starting material (i.e. the carbohydrate or saccharide).

The process of the present invention is carried out in the presence of a solvent. The solvent may be water or a C₁ to C₆ alcohol or polyalcohol (including sugar alcohols), ethers, and other suitable organic compounds or mixtures thereof. Preferred C₁ to C₆ alcohols include methanol, ethanol, 1-propanol and iso-propanol. Polyalcohols of use include glycols, particularly products of the hydrogenation/retro-aldol reaction, glycerol, erythritol, threitol, sorbitol and mixtures thereof. Preferably, the solvent comprises water.

In the process for the preparation of MEG from starting material comprising one or more saccharide, the feed comprising the starting material in a solvent is reacted in the presence of a retro-aldol catalyst composition in a first reaction zone. Said retro-aldol catalyst composition preferably comprises one or more compound, complex or elemental material comprising tungsten, molybdenum, vanadium, niobium, chromium, titanium or zirconium. More preferably the retro-aldol catalyst composition comprises one or more material selected from the list consisting of tungstic acid, molybdic acid, ammonium tungstate, ammonium metatungstate, ammonium paratungstate, silver tungstate, zinc tungstate, zirconium tungstate, tungstate compounds comprising at least one Group 1 or 2 element, metatungstate compounds comprising at least one Group 1 or 2 element, paratungstate compounds comprising at least one Group 1 or 2 element, heteropoly compounds of tungsten including group 1 phosphotungstates, heteropoly compounds of molybdenum, tungsten oxides, molybdenum oxides, vanadium oxides, metavanadates, chromium oxides, chromium sulfate, titanium ethoxide, zirconium acetate, zirconium carbonate, zirconium hydroxide, niobium oxides, niobium ethoxide, and combinations thereof. The metal component is in a form other than a carbide, nitride, or phosphide. Preferably, the retro-aldol catalyst composition comprises one or more compound, complex or elemental material selected from those containing tungsten or molybdenum.

The retro-aldol catalyst composition may be present as a heterogeneous or a homogeneous catalyst composition. In one embodiment, the retro-aldol catalyst composition is heterogeneous and is supported in the first reaction zone. In a preferred embodiment, the retro-aldol catalyst composition is homogeneous with respect to the reaction mixture. In this embodiment, the retro-aldol catalyst composition and any components contained therein, may be fed into the first reaction zone as required in a continuous or discontinuous manner during the process for the preparation of MEG.

Also, in this embodiment, in the process for the preparation of MEG from starting material comprising one or more saccharide, catalyst composition may remain in the intermediate stream and also be present in the second reaction zone and the product stream. Homogeneous retro-aldol catalyst composition may then be separated from at least a portion of the product stream provided for separation and purification of the glycols contained therein. Homogeneous retro-aldol catalyst composition separated from this stream may then be recycled to the first reaction zone.

The weight ratio of the retro-aldol catalyst composition (based on the amount of metal in said composition) to sugar feed is suitably in the range of from 1:1 to 1:1000.

The residence time of the feed stream in the first reaction zone is suitably at least 0.1 second and preferably less than 10 minutes, more preferably less than 5 minutes.

The temperature in the first reaction zone is at least 160° C., preferably at least 170° C., most preferably at least 190° C. The temperature in the first reaction zone is at most 270° C., preferably at most 250° C.

The pressure in the first reaction zone is at least 1 MPa, preferably at least 2 MPa, most preferably at least 3 MPa. The pressure in the first reaction zone is preferably at most 25 MPa, more preferably at most 20 MPa, most preferably at most 18 MPa.

Optimal conditions for the production of glycolaldehyde will require a balance of temperature, pressure and residence times. Such conditions will tend to result in the incomplete conversion of the saccharides present, leading to the presence of one or more monosaccharides.

Concentrations and conditions can be adjusted to control the saccharide conversion. Saccharide conversion in the first reaction zone is at least 10%, preferably at least 20%, more preferably at least 30%. Saccharide conversion in the first reaction zone is preferably at most 99%, more preferably at most 95%, even more preferably at most 90%.

Optionally, the feed stream comprising said starting material in a solvent is contacted with the retro-aldol catalyst composition in the presence of hydrogen.

The intermediate process stream will comprise glycolaldehyde and one or more monosaccharide in a solvent.

The monosaccharides in the process stream comprising glycolaldehyde and one or more monosaccharide in a solvent will preferably comprise at least glucose. C₄ monosaccharides such as erythrose and threose may also be present. Other saccharides, such as oligosaccharides may also be present in this stream.

The process stream comprising glycolaldehyde and one or more monosaccharide in a solvent, particularly in the case of the intermediate process stream will also comprise other reactive intermediates in the reaction of saccharides to glycols. These intermediates, in the absence of hydrogenation, mainly comprise saturated and unsaturated ketones and aldehydes. Such intermediates include, but are not limited to glycolaldehyde, pyruvaldehyde, dihydroxyacetone, glyceraldehyde, hydroxyacetone, erythrose, threose, 1-hydroxy-3,4-butanedione, 1-hydroxy-2-butanone-3-ene, 1-hydroxy-2-butanone, 1,2,3-trihydroxy-5,6-hexanedione and 1-hydroxy-2-hexanone. Highly unsaturated intermediates might polymerise, reducing the yield desired products.

Said process stream comprising glycolaldehyde and one or more monosaccharide in a solvent may also comprise sulfur-containing contaminants, depending on the source of said process stream. If present, such sulfur-containing contaminants are typically present in the range of at most 1000 ppmw (based on the amount of sulfur, considered as the element, in the starting material (i.e. the carbohydrate or saccharide). Preferably the sulfur-containing contaminants are present in the range of at most 600 ppmw. If present, said sulfur-containing contaminants are typically present in the range of at least 10 ppmw (based on the amount of sulfur, considered as the element, in the starting material (i.e. the carbohydrate or saccharide).

The hydrogenation catalyst composition is preferably heterogeneous and is retained or supported within the reactor. Further, said hydrogenation catalyst composition also preferably comprises one or more materials selected from transition metals from groups 8, 9 or 10 or compounds thereof, with catalytic hydrogenation capabilities.

More preferably, the hydrogenation catalyst composition comprises one or more metals selected from the list consisting of iron, cobalt, nickel, ruthenium, rhodium, palladium, iridium and platinum. This metal or metals may be present in elemental form or as compounds. It is also suitable that this component is present in chemical combination with one or more other ingredients in the hydrogenation catalyst composition. It is required that the hydrogenation catalyst composition has catalytic hydrogenation capabilities and it is capable of catalysing the hydrogenation of material present in the reactor.

In one embodiment, the hydrogenation catalyst composition comprises metals supported on a solid support. In this embodiment, the solid supports may be in the form of a powder or in the form of regular or irregular shapes such as spheres, extrudates, pills, pellets, tablets, monolithic structures. Alternatively, the solid supports may be present as surface coatings, for examples on the surfaces of tubes or heat exchangers. Suitable solid support materials are those known to the skilled person and include, but are not limited to aluminas, silicas, zirconium oxide, magnesium oxide, zinc oxide, titanium oxide, carbon, activated carbon, zeolites, clays, silica alumina and mixtures thereof.

Alternatively, the heterogeneous hydrogenation catalyst composition may be present as Raney material, such as Raney nickel or Raney ruthenium, preferably present in a pelletised form.

The heterogeneous hydrogenation catalyst composition is suitably preloaded into the reactor before the reaction is started.

The process stream is contacted with hydrogen in the presence of said hydrogenation catalyst composition at a temperature of no more than 150° C. and for a residence time of no more than 90 minutes. Preferably, the process stream is an intermediate stream in a process for the preparation of monoethylene glycol from starting material comprising one or more saccharides as indicated above.

The process stream may be reduced in temperature by any suitable method known in the art. Typical methods include, but are not limited to flashing (i.e. reducing the pressure), quenching (mixing with a lower temperature stream) and heat exchange, preferably with high heat transfer area per unit volume.

In this embodiment, the amount of hydrogenation catalyst composition (based on the amount of metal in said composition) as a percentage of the total reaction mixture is in the range of from 0.001 to 10 wt %.

The residence time for which the stream is contacted with hydrogen in the presence of said hydrogenation catalyst composition is preferably at least 1 second, more preferably at least 1 minute, even more preferably at least 30 minutes. Said residence time is no more than 90 minutes.

The process stream, or intermediate process stream, is contacted with hydrogen in the presence of the hydrogenation catalyst composition at a temperature of no more than 150° C. Preferably, the temperature is no more than 120° C., even more preferably no more than 100° C. Also preferably, the temperature is at least 20° C., preferably at least 50° C.

The process stream, or intermediate process stream, is contacted with hydrogen in the presence of the hydrogenation catalyst composition and the pressure in the reactor is generally at least 1 MPa, preferably at least 2 MPa, more preferably at least 3 MPa. The pressure in the reactor is generally at most 25 MPa, more preferably at most 20 MPa, even more preferably at most 18 MPa.

A product stream comprising glycols and one or more monosaccharide is withdrawn from the second reaction zone. Said glycols preferably comprise at least MEG, MPG and 1,2-BDO. The monosaccharides in this process stream preferably comprise one or more monosaccharides selected from glucose, erythrose and threose. Even more preferably the one or more monosaccharide comprises glucose. The product stream may suitably also contain solvent, by-products and catalyst composition.

Preferably, the ratio of the one or more monosaccharide to C₄-C₆ sugar alcohols present in the product stream is at least 2:1, more preferably at least 5:1, even more preferably at least 10:1.

The hydrogenation step and, optionally, the retro-aldol step of the process of the present invention take place in the presence of hydrogen. Preferably, both steps (if carried out) take place in the absence of air or oxygen. In order to achieve this, it is preferable that the atmosphere under which the process takes place (e.g. in the reaction zones) be evacuated and replaced with first an inert gas, e.g. nitrogen or argon, and then hydrogen repeatedly, after loading of any initial contents, before the reaction starts.

A portion of the product stream is provided for separation and purification of the glycols contained therein. Steps for purification and separation may include solvent removal, catalyst separation, distillation and/or extraction in order to provide the desired glycol products.

In the embodiment wherein first and second reaction zones are present, said reaction zones are physically distinct from one another. Each reaction zone may be an individual reactor or reactor vessel or the zones may be contained within one reactor vessel.

In a preferred embodiment of the invention, the feed stream comprising the starting materials is provided to an external recycle loop of a reactor vessel, via an inlet in said external recycle loop, and is contacted with the homogeneous retro-aldol catalyst composition within said external recycle loop. Thus, the external recycle loop is the first reaction zone.

In this embodiment, the intermediate stream is then provided from the external recycle loop into the reactor vessel wherein it is contacted with hydrogen in the presence of a hydrogenation catalyst composition. Thus the reactor vessel operates as the second reaction zone. The product stream is then withdrawn from the reactor vessel and a portion of it is removed, via an outlet, for purification and separation of the glycols contained therein. The remainder of the product stream is then recycled to the reactor vessel via the external recycle loop.

The remainder of the product stream will suitably be re-heated before recycling to the first reaction zone. Preferably, this is done by a fast heating method in order to minimise sugar degradation. Suitable methods include, but are not limited to live steam injection and heat exchange, preferably using high heat transfer area per unit volume.

Hydrogen may suitably be removed from the product stream withdrawn from the reactor vessel, preferably by flashing. Said hydrogen may then be recycled to the reactor vessel.

Also in this embodiment, the inlet in the external recycle loop through which the feed stream is provided is downstream of the outlet through which a portion of the product stream is withdrawn. Other inlets may also be present in the external recycle loop. A homogeneous retro-aldol catalyst composition containing stream may be supplied separately to the feed stream comprising starting materials. This stream may be provided before or after the feed stream comprising starting materials. A further solvent stream may also be present.

The reactor vessel used in the process for the preparation of MEG from starting material comprising one or more saccharide may operate with a high degree of back-mixing or may operate in an essentially plug flow manner.

In a reactor vessel operating with a high degree of back mixing, mixing should be carried out to such an extent that the concentrations of the materials in the reactor are relatively consistent throughout. The degree of mixing for a reactor is measured in terms of a Péclet number. An ideally-stirred tank reactor vessel would have a Péclet number of 0. In this embodiment, wherein the reactor vessel operates with a high degree of mixing, the Péclet number is preferably at most 0.4, more preferably at most 0.2, even more preferably at most 0.1, most preferably at most 0.05.

It will be clear to the skilled person, however, that concentrations of any materials may be considerably higher or lower in the immediate vicinity of an inlet to the reactor vessel. Suitable reactor vessels include those considered to be continuous stirred tank reactors. Examples include slurry reactors ebbulated bed reactors, jet flow reactors, mechanically agitated reactors and (slurry) bubble columns. The use of these reactor vessels allows dilution of the reaction mixture to an extent that provides high degrees of selectivity to the desired glycol product (mainly ethylene and propylene glycols).

In a reactor vessel operating with essentially a plug flow, all of the feed stream moves with the same radially uniform velocity and, therefore, has the same residence time. The concentration of the reactants in the plug flow reactor vessel will change as it progresses through the reactor vessel. Although the reaction mixture preferably essentially completely mixes in radial direction and preferably does essentially not mix in the axial direction (forwards or backwards), in practice some mixing in the axial direction (also referred to as back-mixing) may occur. Suitable reactor vessels operating with essentially plug flow include, but are not limited to, tubular reactors, pipe reactors, falling film reactors, staged reactors, packed bed reactors and shell and tube type heat exchangers.

A plug flow reactor vessel may, for example, be operated in the transition area between laminar and turbulent flow or in the turbulent area, such that a homogenous and uniform reaction profile is created.

A plug flow may for example be created in a tubular reactor vessel. It may also be created in a compartmentalized tubular reactor vessel or in another reactor vessel or series of reactor vessels having multiple compartments being transported forward, where preferably each of these compartments are essentially completely mixed. An example of a compartmentalized tubular reactor vessel operated at plug flow may be a tubular reactor vessel comprising a screw.

Preferably a Péclet number of at least 3, more preferably at least 6, and still more preferably at least 20, most preferably at least 100, is maintained within the plug flow reactor vessel.

In one embodiment of the invention, the portion of the product stream which has been removed for separation and purification of the glycols contained therein may be subjected to further reaction in a finishing reactor in order to ensure that the reaction has gone to completion.

Preferably said finishing reactor operate in an essentially plug flow manner. Further hydrogenation catalyst composition may be present in said finishing reactor. In the embodiment wherein the retro-aldol catalyst composition is homogeneous with respect to the reaction mixture, said retro-aldol catalyst composition will be present in the portion of the product stream which has been removed from the reactor system.

### Detailed Description of the Drawings

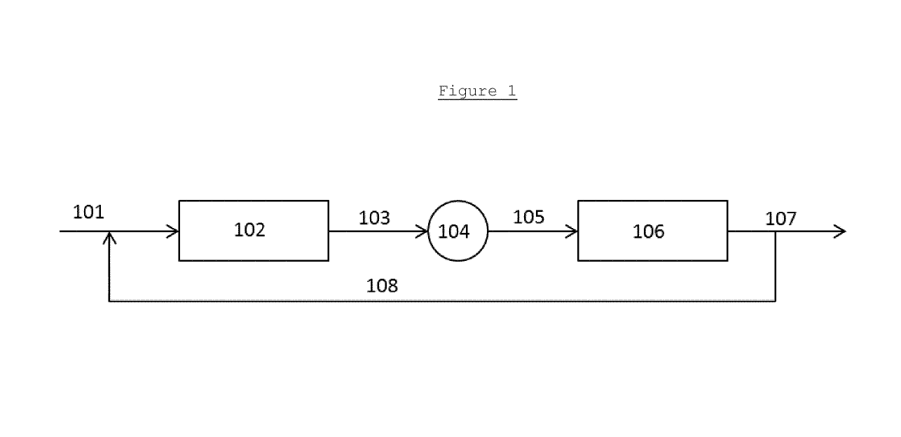

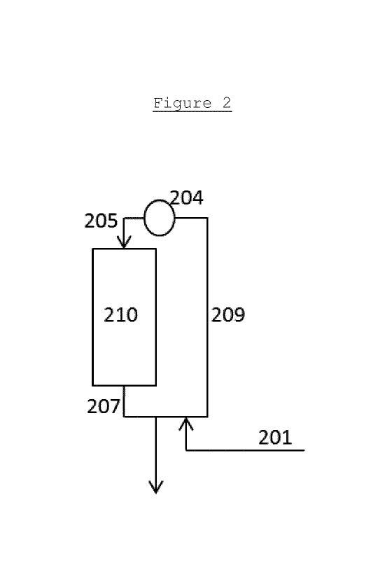

In these Figures, the first digit of each reference number refers to the Figure number (i.e. 1XX for FIG. 1 and 2XX for FIG. 2). The remaining digits refer to the individual features and the same features are provided with the same number in each Figure. Therefore, the same feature is numbered 104 in FIG. 1 and 204 in FIG. 2.

FIG. 1 illustrates a non-limiting, embodiment of the present invention.

Feed stream 101 is provided to a first reaction zone 102, wherein it is contacted with a retro-aldol catalyst at a temperature in the range of from 160 to 270° C. The resultant intermediate stream 103 comprising glucose and glycolaldehyde is cooled in cooler 104 to provide a cooled intermediate stream 105. Said cooled intermediate stream 105 is provided to a second reaction zone 106 and is contacted therein with hydrogen in the presence of a hydrogenation catalyst composition at a temperature of no more than 150° C. and for a residence time of no more than 90 minutes.

The product stream 107 is then withdrawn from the second reaction zone 106 and a portion of it is removed, via an outlet, for purification and separation of the glycols contained therein. The remainder 108 of the product stream is then recycled to the first reaction zone 102.

Hydrogen may also be removed from the product stream 107, preferably by flashing. Said hydrogen may then be recycled to the process, for example to the second reaction zone.

FIG. 2 illustrates an embodiment wherein the first reaction zone takes the form of an external recycle loop 209 of a reactor vessel 210 which forms the second reaction zone. In this embodiment, the reactor vessel operates in an essentially plug flow manner.

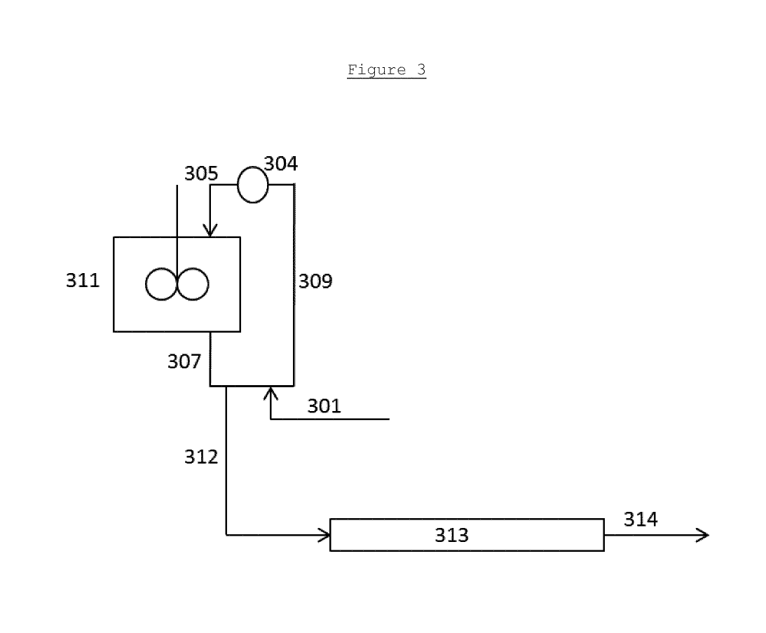

A similar embodiment is illustrated in FIG. 3. However, in FIG. 3, the reactor vessel 310 is a stirred reactor vessel. In this embodiment, the portion 312 of the product stream 307 removed for purification and separation of the glycols contained therein is first subjected to further reaction in a finishing reactor 313, before the purification and separation of the resultant stream 314.

The present invention is further illustrated in the following Examples.

### EXAMPLES

Hastelloy C batch autoclaves (75 ml), with magnetic stir bars, were used to screen various conditions and catalyst systems.

Known weights of catalysts, 1 wt % glucose (when used) and 1 wt % glycolaldehyde were added to the autoclaves along with 30 ml of the solvent (typically water).

If the catalysts or feedstocks were present as slurries or solutions, the total volume of those as well as the solvent was kept at 30 ml.

### Examples 1 to 6

### Methodology

Glucose (0.3 g) and glycolaldehyde (0.3 g) were dissolved in 30 ml of water. Hydrogenation catalyst was also added to the solution. The loaded autoclave was then purged three times with nitrogen, followed by hydrogen purge.

The hydrogen pressure was then raised to ˜14 MPa of hydrogen and the autoclave was sealed and left stirring overnight to do a leak test.

The next morning the autoclave was depressurised to the target hydrogen pressure (10.1 MPa) at room temperature, and closed. The temperature was then ramped to the target run temperature as a fast ramp.

The autoclave was held at the target temperature for known durations of time (15 min, 30 min or 75 min), while both the temperature and pressure were monitored. After the required run time had elapsed, the heating was stopped, and the reactor was cooled down to room temperature, depressurised, purged with nitrogen and then opened.

The contents of the autoclave were then analyzed via Gas Chromatography (GC) or High Pressure Liquid Chromatography (HPLC) after being filtered. The yield of MEG was measured as wt % basis of the glycolaldehyde loaded (maximum theoretical yield ˜104%), while the yield of sorbitol was measured as a wt % basis the glucose loaded.

Table 1 provides details of the reaction conditions and results of Examples 1 to 6:

TABLE 1

| | | | | | MEG, | MEG, | | |
|---|---|---|---|---|---|---|---|---|
| | | | | | wt % | wt % | | |
| | | | Run | Run | GC | HPLC | Glucose, | Sorbitol, |
| | | Catalyst | temp | Length, | Basis | | wt % HPLC | wt % HPLC |
| | Catalyst | Amount g | ° C. | min | Glycolaldehyde | | Basis Glucose | |
| 1 | Raney Ni | 0.02 | 40 | 30 | 104.2 | 96.7 | 92.4 | 3.7 |
| 2 | Raney Ni | 0.02 | 40 | 75 | 104.7 | 97.2 | 91 | 5.7 |
| 3 | Raney Ni | 0.02 | 70 | 30 | 100.9 | 99.0 | 92.1 | 6.7 |
| 4 | Raney Ni | 0.02 | 70 | 75 | 104.9 | 97.6 | 89 | 8.2 |
| 5 | Raney Ni | 0.02 | 100 | 30 | 102.0 | 95.2 | 72.3 | 22.5 |
| 6 | Raney Ni | 0.02 | 100 | 75 | 101.8 | 94.3 | 48.3 | 46.1 |

Examples 1 to 6 show that glycolaldehyde can be quantitatively converted to MEG, while at temperatures lower than 70 deg C, less than -10% of the glucose gets hydrogenated to sorbitol. Restricting the residence time of the reaction also restricts the amount of glucose that is hydrogenated to sorbitol.

### Examples 7 and 8

The same methodology as described for Examples 1 to 6 was used but different hydrogenation catalysts were used. The target temperature was 70° C. and run length was 30 min. Table 2 shows the different catalyst systems and the results.

TABLE 2

| | | | MEG | | Glucose | Sorbitol |
|---|---|---|---|---|---|---|
| | | | wt % | MEG, wt % | wt % | wt % |
| | | Catalyst | GC | HPLC | HPLC | HPLC |
| | Catalyst | Amount g | Basis Glycolaldehyde | | Basis Glucose | |
| 3 | Raney Ni | 0.02 | 104.2 | 96.7 | 92.4 | 3.7 |
| 7 | Raney Ru | 0.02 | 104.7 | 97.2 | 91 | 5.7 |
| 8 | 1.2 wt % | 0.02 | 100.9 | 99.0 | 92.1 | 6.7 |
| | Ru on | | | | | |
| | carbon | | | | | |

Examples 3, 7 and 8 show that, using different catalysts, glycolaldehyde is quantitatively converted to MEG in the presence of glucose.

### Examples 9 to 12

The same methodology was used as in previous examples but with different hydrogen pressures as indicated in Table 3. The target temperature for each of these examples was 100 ° C. In each case the run length was 30 min and 0.02 g of Raney Ni was used as the hydrogenation catalyst.

TABLE 3

| | | | MEG | MEG | | |
|---|---|---|---|---|---|---|
| | Pressure at | Actual Avg | wt % | wt % | | Sorbitol |
| | room | Run | GC | HPLC | Glucose | wt % |
| | temperature, | Pressure, | Basis | | wt % HPLC | HPLC |
| | MPa | MPa | Glycolaldehyde | | Basis Glucose | |
| 9 | 0.27 | 0.35 | 91.3 | 92 | 94.5 | 1.9 |
| 10 | 0.45 | 0.58 | 102.2 | 99.6 | 90.6 | 8 |
| 11 | 2.55 | 3.02 | 94.2 | 94.6 | 93.4 | 4.9 |
| 12 | 5 | 6.06 | 101.6 | 98.9 | 87.1 | 12.1 |
| 5 | 10.1 | 12.48 | 102.0 | 95.2 | 72.3 | 22.5 |

Table 3 shows that even at very low pressure more than 90% of the glycolaldehyde is hydrogenated to MEG in the presence of glucose.

### Examples 13 to 18

Further examples were run with a range of catalysts, catalyst loadings, temperatures and residence times. The results are shown in Table 4.

TABLE 4

| | | | | | MEG | MEG, | | |
|---|---|---|---|---|---|---|---|---|
| | | | | | wt % | wt % | Glucose | Sorbitol |
| | | | Run | Run | GC | HPLC | wt % | wt % |
| | | Catalyst | temp | Length, | Basis | | HPLC | HPLC |
| | Catalyst | Amount g | ° C. | min | Glycolaldehyde | | Basis Glucose | |
| 13 | Raney Ni | 0.005 | 70 | 30 | 98.7 | 97.9 | 95.7 | 2.1 |
| 3 | Raney Ni | 0.02 | 70 | 30 | 100.9 | 99.0 | 92.1 | 6.7 |
| 14 | Raney Ni | 0.02 | 70 | 30 | 104.0 | 97.5 | 85.4 | 11.9 |
| 15 | Raney Ni | 0.005 | 100 | 30 | 85.2 | 85 | 97.4 | 2.6 |
| 5 | Raney Ni | 0.02 | 100 | 30 | 102.0 | 95.2 | 72.3 | 22.5 |
| 16 | Raney Ru | 0.005 | 70 | 30 | 103.6 | 100.1 | 96.6 | 3.5 |
| 7 | Raney Ru | 0.02 | 70 | 30 | 103.7 | 100.6 | 84.8 | 15.8 |
| 17 | Raney Ru | 0.005 | 100 | 30 | 104.8 | 101.8 | 94.6 | 7.0 |
| 18 | Raney Ru | 0.02 | 100 | 30 | 104.6 | 101.1 | 44.4 | 56.1 |

### Examples 19 to 22

The same methodology was used as in previous examples but with 1 wt % glycolaldehyde (no glucose) with and without 10 ppm of S from methionine as the representative S contaminant. The run conditions and results are shown in Table 5.

TABLE 5

| | | | Run | Run | | MEG, |
|---|---|---|---|---|---|---|
| | | Catalyst | temp | Length, | S, | wt % |
| | Catalyst | Amount g | ° C. | min | ppm | GC |
| 19 | Raney Ni | 0.02 | 80 | 30 | 10 | 102.1 |
| 20 | Raney Ni | 0.02 | 120 | 30 | 10 | 98.9 |

Examples 19 and 20 clearly show that at lower temperatures of 80° C. and 120° C., the hydrogenation catalyst (Raney Ni) is not affected by the presence of 10 ppm of S and that almost quantitative conversion of glycolaldehyde to MEG takes place.

### Examples 21 to 24

The same methodology was used as in previous examples but with 1 wt % glycolaldehyde (no glucose) with 10 ppm of S from methionine as the representative S contaminant with various hydrogenation catalysts. The run conditions and results are shown in Table 6.

TABLE 6

| | | | Run | Run | | MEG, |
|---|---|---|---|---|---|---|
| | | Catalyst | temp | Length, | S, | wt % |
| | Catalyst | Amount g | ° C. | min | ppm | GC |
| 19 | Raney Ni | 0.02 | 80 | 30 | 10 | 102.1 |
| 21 | Raney Ru | 0.02 | 80 | 30 | 10 | 99.3 |
| 22 | 1.2 wt % Ru on carbon | 0.08 | 80 | 30 | 10 | 93.4 |
| 20 | Raney Ni | 0.02 | 120 | 30 | 10 | 98.9 |
| 23 | Raney Ru | 0.02 | 120 | 30 | 10 | 101.9 |
| 24 | 1.2 wt % Ru on carbon | 0.08 | 120 | 30 | 10 | 93.0 |

Table 6 shows that almost quantitative hydrogenation of glycolaldehyde to MEG was obtained with Raney Ni and Raney Ru in the presence of Sulfur contaminants. Slightly lower, but still acceptable compared to expected yields with this catalyst, yields were obtained with 1.2 wt % Ru on carbon.

1. A process for the selective hydrogenation of glycolaldehyde in a process stream comprising glycolaldehyde and one or more monosaccharide in a solvent, said process comprising contacting the process stream with hydrogen in the presence of a hydrogenation catalyst composition at a temperature of no more than 150° C. and for a residence time of no more than 90 minutes.

2. The process according to claim 1, wherein the process stream comprising glycolaldehyde and one or more monosaccharide also comprises sulfur-containing contaminants in an amount in the range of from 10 to 1000 ppmw.

3. A continuous process for the preparation of monoethylene glycol from starting material comprising one or more saccharides by:
i) contacting a feed stream comprising said starting material in a solvent with a retro-aldol catalyst composition in a first reaction zone at a temperature in the range of from 160 to 270° C. to provide an intermediate process stream comprising one or more monosaccharide and glycolaldehyde;
ii) then contacting said intermediate process stream with hydrogen in the presence of a hydrogenation catalyst composition in a second reaction zone at a temperature of no more than 150° C. and for a residence time of no more than 90 minutes;
iii) withdrawing a product stream comprising glycols and one or more monosaccharide from the second reaction zone;
iv) providing a portion of said product stream for separation and purification of the glycols contained therein; and
v) recycling the rest of the product stream to the first reaction zone.

4. The process according claim 3, wherein the process stream comprising glycolaldehyde and one or more monosaccharide also comprises sulfur-containing contaminants in an amount in the range of from 10 to 1000 ppmw.

5. The process according io claim 3, wherein the starting material comprising one or more saccharides comprises starch, hydrolysed starch or a mixture thereof.

6. The process according to claim 1, wherein the one or more monosaccharide in the process stream comprising glycolaldehyde and one or more monosaccharide in a solvent comprises glucose.

7. The process according to claim 1, wherein the process stream comprising glycolaldehyde and one or more monosaccharide in a solvent also comprises a homogeneous retro-aldol catalyst composition.

8. The process according to claim 1, wherein the hydrogenation catalyst composition comprises one or more materials selected from transition metals from groups 8, 9 or 10, or compounds thereof with catalytic hydrogenation capabilities.

9. The process according to claim 1, wherein the process stream is contacted with hydrogen in the presence of a hydrogenation catalyst composition at a temperature of no more than 100° C.

10. The process according to claim 3, wherein the ratio of the one or more monosaccharide to C₄-C₆ sugar alcohols present in the product stream is at least 2:1.

11. The process according to claim 3, wherein the intermediate process stream is reduced in temperature before step ii) by a process selected from flashing, quenching and heat exchange using high heat transfer area per unit volume.

12. The process according to claim 3, wherein the rest of the product stream that is recycled to the first reaction zone is heated by live steam injection or by heat exchange, preferably using high heat exchange transfer are per unit volume.
